(19) United States
(12) Patent Application Publication
(10) Pub. No.: US 2026/0055044 A1
(43) Pub. Date: Feb. 26, 2026

(54) ENANTIOMERIC ENRICHMENT OF L-LACTIC ACID OR SALT THEREOF

(71) Applicant: TRIPLEW LTD., Netanya (IL)

(72) Inventors: Tal SHAPIRA, Brasschaat (BE); Nitsan PAPO, Hod Hasharon (IL); Dina PINSKY, Jerusalem (IL)

(21) Appl. No.: 18/993,132

(22) PCT Filed: Jul. 26, 2023
(86) PCT No.: PCT/IL2023/050775

§ 371 Date: Jan. 10, 2025

Related U.S. Application Data
(60) Provisional application No. 63/392,508, filed on Jul. 27, 2022.

(51) Int. Cl. C07C 51/41; C07C 51/02; C07C 51/43; C07C 51/47; C08G 63/08

(52) U.S. Cl. CPC C07C 51/418; C07C 51/02; C07C 51/43; C07C 51/47; C08G 63/08

(57) ABSTRACT

The present invention relates to processes for the enantiomeric enrichment of lactic acid or salt thereof obtained from waste recycling.

### FIELD OF THE INVENTION

The present invention relates to processes for enriching the enantiomeric purity of lactic acid derived from recycled waste.

### BACKGROUND OF THE INVENTION

Polylactic acid constitutes an alternative to petroleum-derived plastics. Accordingly, its use in the manufacture of products such as food packaging, disposables, and fibers in the textile and hygiene products industries, is continuously growing. In addition, polylactic acid is a biodegradable polymer and is therefore used in a variety of biomedical applications including the production of suture threads, bone fixation screws, and devices for drug delivery.

Polylactic acid is formed by the polymerization of lactic acid monomers. Lactic acid has a chiral carbon atom and therefore exists in two enantiomeric forms, D- and L-lactic acid. In order to produce polylactic acid that is suitable for industrial applications, the D- or L-lactic acid entering the production process must be highly purified to meet the specification required for polymerization and must contain primarily (above ˜98%) one lactic acid enantiomer to produce either PLLA (from L-lactic acid) or PDLA (from D-lactic acid).

Lactic acid fermentation has been gaining interest in recent years due to the ability of certain bacteria to produce only one discreet lactic acid enantiomer (L or D). The conventional fermentation process is typically based on anaerobic fermentation of a carbohydrate source (e.g., starch and sugars from corn or sugar cane) by lactic acid-producing microorganisms, which produce lactic acid as the major metabolic end product of carbohydrate fermentation. For production of polylactic acid, the lactic acid generated during the fermentation is separated from the fermentation broth and purified by various processes, and the purified lactic acid is then subjected to polymerization. Any endogenous D-lactic acid that may be present in the fermentation broth needs to be removed in order to produce optically pure polylactic acid. WO 2017/122197, assigned to the Applicant of the present invention, discloses dual action lactic-acid (LA)-utilizing bacteria genetically modified to secrete polysaccharide-degrading enzymes such as cellulases, hemicellulases, and amylases, useful for processing organic waste both to eliminate lactic acid present in the waste and degrade complex polysaccharides. WO 2020/208635, assigned to the Applicant of the present invention, discloses systems and methods for processing organic waste, particularly mixed food waste, using a D-lactate oxidase, which eliminates D-lactic acid that is present in the organic waste.

Polylactic acid waste can be recycled by landfilling, composting, anaerobic digestion (biogas production), incineration or chemical hydrolysis back into lactic acid monomers or lactides. One of the common forms of polylactic acid on the market is the copolymer PDLLA (poly (D-L-)lactic acid), predominantly composed of PLLA (made from L-lactic acid), and minor amounts of PDLA (made from D-lactic acid). A significant portion of the polylactic acid plastics present on the market contains a small amount of PDLA that when hydrolyzed, releases D-lactic acid. The hydrolyzed material may also contain unknown amounts of D-lactic acid formed by racemization during the hydrolysis.

An optical purity of over ˜98% is typically required for both D-lactic acid and L-lactic acid entering the production process of new polylactic acid. Therefore, the use of lactic acid from fermentation processes or hydrolysis of polylactic acid should address the issue of isomer separation which may be expensive, typically involving liquid or solid enantioselective membranes or high-performance liquid chromatography (HPLC).

Hydrolysis of polylactic acid using an alkaline material such as sodium hydroxide has been studied (Cam, Hyon and Ikada (1995) Biomaterials, 16(11): 833-43); Chauliac (2013) “Development of a thermochemical process for hydrolysis of polylactic acid polymers to L-lactic acid and its purification using an engineered microbe” Ph.D. thesis, University of Florida, UMI Number: 3583516; Wadsö and Karlsson (2013) Polymer Degradation and Stability, 98(1): 73-78.

Siparsky, Voorhees, and Miao (1998) Journal of environmental polymer degradation, 6(1): 31-41, report the hydrolysis of polylactic acid (PLA) and polycaprolactone (PCL) in aqueous acetonitrile solutions.

Xu, Crawford and Gorman (2011) Macromolecules, 44(12): 4777-4782, report the effects of temperature and pH on the degradation of poly(lactic acid) brushes.

Elsawy et al. (2017) Renewable and Sustainable Energy Reviews, 79: 1346-1352, review the hydrolytic degradation of polylactic acid (PLA) and its composites.

Motoyama et al. (2007) Polymer Degradation and Stability, 92(7): 1350-1358, report the effects of MgO catalyst on depolymerization of poly-L-lactic acid to L,L-lactide.

WO 2015/112098 discloses a process for manufacturing lactide from plastics having polylactic acid (PLA-based plastics) that comprises preparing PLA-based plastics, accelerating decomposition of polylactic acid in the plastics by alcoholysis or hydrolysis to provide low molecular weight polylactic acid, and thermal decomposition of the low molecular weight polylactic acid to provide lactide. Also, the process further comprises minimizing the size of the PLA-based plastics after the preparation step, and purifying lactide after thermal decomposition of the low molecular weight polylactic acid.

U.S. Pat. No. 7,985,778 discloses a method for decomposing and reclaiming synthetic resin having ester bond in composition structure thereof, by conducting hydrolysis treatment and then separation collection treatment. In the hydrolysis treatment, an article containing synthetic resin to be decomposed and reclaimed is exposed to water vapor atmosphere filled under saturation water vapor pressure at treatment temperature at or below melting point of the synthetic resin. The synthetic resin in article to be treated is hydrolyzed by water vapor generated at the treatment temperature, to generate decomposition product before polymerizing to the synthetic resin containing an ester bond. The separation collection treatment is treatment in which the decomposition product generated by the hydrolysis treatment is separated into liquid component and solid component to be collected individually.

U.S. Pat. No. 8,614,338 discloses a method for the stereospecific chemical recycling of a mixture of polymers based on polylactic acid PLA, in order to reform the monomer thereof or one of the derivatives thereof. The method comprises a step of putting the mixture of polymers in suspension in a lactic ester able to dissolve the PLA fraction followed by a separation firstly of the lactic ester, the PLA and other dissolved impurities and secondly the mixture of other polymers and impurities that are insoluble. The solution containing the PLA thus obtained is then subjected to a catalytic depolymerization reaction by transesterification in order to form oligoesters. The depolymerization reaction by transesterification is then stopped at a given moment and the residual lactic ester separated. The oligoester thus obtained then undergoes a cyclisation reaction in order to produce lactide that will finally be purified stereospecifically so as to obtain a fraction of purified lactide having a meso-lactide content of between 0.1% and 40%.

U.S. Pat. Nos. 8,431,683 and 8,481,675 disclose a process for recycling a polymer blend necessarily containing PLA, comprising grinding, compacting, dissolving in a solvent of PLA, removing the undissolved contaminating polymers, alcoholysis depolymerization reaction and purification steps.

U.S. Pat. No. 8,895,778 discloses depolymerization of polyesters such as post-consumer polylactic acid. Ultrasonic induced implosions can be used to facilitate the depolymerization. Post-consumer PLA was exposed to methanol as the suspension media in the presence of organic or ionic salts of alkali metals such a potassium carbonate and sodium hydroxide as depolymerization catalysts to provide high quality lactic acid monomers in high yield.

U.S. 2018/0051156 discloses a method for enhancing/accelerating the depolymerization of polymers (e.g., those containing hydrolyzable linkages), the method generally involves contacting a polymer comprising hydrolyzable linkages with a solvent and an alcohol to give a polymer mixture in which the polymer is substantially dissolved, wherein the contacting is conducted at a temperature at or below the boiling point of the polymer mixture. A resulting depolymerized polymer can be separated therefrom (including, e.g., monomers and/or oligomers). Such methods can be conducted under relatively mild temperature and pressure conditions. In some embodiments, the polymer is poly(lactic acid).

WO 2021/165964, assigned to the Applicant of the present invention, discloses industrial fermentation for the production of lactic acid from organic waste combined with chemical recycling of polylactic acid to obtain lactic acid at high yields.

WO 2022/157768, assigned to the Applicant of the present invention, discloses a process for the crystallization of high purity magnesium L-lactate from decomposed organic waste.

WO 2023/012791, assigned to the Applicant of the present invention, discloses a process for producing magnesium L-lactate in high enantiomeric purity and low amounts of non-lactate impurities and enriching the enantiomeric purity of recycled lactate salt containing an enantiomeric mixture of L- and D-lactate.

There remains an unmet need for economical and reliable processes for generation of enantiomerically pure L-lactic acid/lactate salt from decomposed waste.

### SUMMARY OF THE INVENTION

The present invention provides a process for enriching the enantiomeric purity of lactic acid obtained, for example, from lactic acid fermentation of organic waste and/or PLA waste chemical recycling. The present invention further provides a process for obtaining L-lactate salt with enriched enantiomeric purity.

The present invention is based, in part, on the unexpected finding that the enantiomeric purity of L-lactic acid can be enhanced by selective precipitation of L-lactate salt from an enantiomeric mixture containing D-and L-lactic acid. The present invention therefore enables the recycling of waste from various sources including waste that contains endogenous D-lactic acid monomers while obviating the need for D-lactic acid-utilizing bacteria or enzymes to eliminate D-lactic acid. The present invention advantageously produces L-lactate salt in enantiomeric purity of over 98% which can be used for the commercial production of PLA with no additional processing for isomer separation.

Disclosed herein for the first time is the selective precipitation of L-lactate salt from an enantiomeric mixture of D- and L-lactic acid originated from organic waste. The selective precipitation results in enrichment in L-lactic acid/lactate monomers for subsequent use.

According to a first aspect, the present invention provides a process for enriching L-lactic acid enantiomer or salt thereof, the process comprising the steps of:
(a) obtaining decomposed organic waste comprising an enantiomeric mixture of D- and L-lactic acid at a concentration of about 100 to about 1,100 g/L, wherein the enantiomeric mixture comprises 10% D-lactic acid or less;
(b) contacting the enantiomeric mixture with an alkaline substance comprising divalent cations in an amount of about 50 to about 120 equivalent % based on the number of carboxyl groups present in the enantiomeric mixture; and
(c) precipitating L-lactate salt with said divalent cations thereby obtaining L-lactate salt with enriched enantiomeric purity as compared to the initial enantiomeric mixture,
   wherein step (b) and/or (c) are performed at a temperature in the range of about 40° C. to about 100° C., including each value within the specified ranges.

According to another aspect, the present invention provides a process for producing enantiomerically enriched L-lactate salt, the process comprising the steps of:
(a) obtaining decomposed organic waste comprising an enantiomeric mixture of D- and L-lactic acid at a concentration of about 100 to about 1,100 g/L, wherein the enantiomeric mixture comprises 10% D-lactic acid or less;
(b) contacting the enantiomeric mixture with an alkaline substance comprising divalent cations in an amount of about 50 to about 120 equivalent % based on the number of carboxyl groups present in the enantiomeric mixture; and
(c) precipitating L-lactate salt with said divalent cations thereby obtaining L-lactate salt with enriched enantiomeric purity as compared to the initial enantiomeric mixture,
   wherein step (b) and/or (c) are performed at a temperature in the range of about 40° C. to about 100° C., including each value within the specified ranges.

In some embodiments, the enantiomeric mixture comprises 9% D-lactic acid or less. In other embodiments, the enantiomeric mixture comprises 8% D-lactic acid or less. In yet other embodiments, the enantiomeric mixture comprises 7% D-lactic acid or less. In additional embodiments, the enantiomeric mixture comprises 6% D-lactic acid or less. In further embodiments, the enantiomeric mixture comprises 5% D-lactic acid or less. In various embodiments, the enantiomeric mixture comprises 4% D-lactic acid or less. In particular embodiments, the enantiomeric mixture comprises 3% D-lactic acid or less. In certain embodiments, the enantiomeric mixture comprises 3% D-lactic acid or more. In several embodiments, the enantiomeric mixture comprises 4% D-lactic acid or more. In exemplary embodiments, the enantiomeric mixture comprises 5% D-lactic acid or more.

In additional embodiments, step (b) and/or (c) are performed at a temperature in the range of about 45° C. to about 100° C., including each value within the specified range. In other embodiments, step (b) and/or (c) are performed at a temperature in the range of about 50° C. to about 100° C., including each value within the specified range.

According to another aspect, the present invention provides a process for enriching L-lactic acid enantiomer or salt thereof, the process comprising the steps of:
(a) obtaining decomposed organic waste comprising an enantiomeric mixture of D- and L-lactic acid at a concentration of about 100 to about 1,100 g/L. wherein the enantiomeric mixture comprises 6% D-lactic acid or less;
(b) contacting the enantiomeric mixture with an alkaline substance comprising divalent cations; and
(c) precipitating L-lactate salt with said divalent cations thereby obtaining L-lactate salt with enriched enantiomeric purity as compared to the initial enantiomeric mixture,
   wherein step (b) and/or (c) are performed at a temperature in the range of about 50° C. to about 100° C., including each value within the specified ranges.

According to an additional aspect, the present invention provides a process for producing enantiomerically enriched L-lactate salt, the process comprising the steps of:
(a) obtaining decomposed organic waste comprising an enantiomeric mixture of D- and L-lactic acid at a concentration of about 100 to about 1,100 g/L, wherein the enantiomeric mixture comprises 6% D-lactic acid or less;
(b) contacting the enantiomeric mixture with an alkaline substance comprising divalent cations; and
(c) precipitating L-lactate salt with said divalent cations thereby obtaining L-lactate salt with enriched enantiomeric purity as compared to the initial enantiomeric mixture,
   wherein step (b) and/or (c) are performed at a temperature in the range of about 50° C. to about 100° C., including each value within the specified ranges.

In various embodiments, the processes disclosed herein provide enrichment of L-lactic acid enantiomer or salt thereof by 1% or more. In another embodiment, the processes provide enrichment of L-lactic acid enantiomer or salt thereof by 5% or more. In yet another embodiment, the processes provide enrichment of L-lactic acid enantiomer or salt thereof by 10% or more. In particular embodiments, the processes provide enrichment of L-lactic acid enantiomer or salt thereof of up to 10%. In other embodiments, the processes provide reduction in D-lactic acid enantiomer or salt thereof of 10% or more. In yet other embodiments, the processes provide reduction of D-lactic acid enantiomer or salt thereof of 20% or more. In additional embodiments, the processes provide reduction of D-lactic acid enantiomer or salt thereof of 30% or more. In certain embodiments, the processes provide reduction of D-lactic acid enantiomer or salt thereof of 40% or more. In additional embodiments, the processes provide reduction of D-lactic acid enantiomer or salt thereof of 50% or more. In further embodiments, the processes provide reduction of D-lactic acid enantiomer or salt thereof of 60% or more. In exemplary embodiments, the processes provide reduction of D-lactic acid enantiomer or salt thereof of 70% or more. In particular embodiments, the processes provide reduction of D-lactic acid enantiomer or salt thereof of 80% or more. In several embodiments, the processes provide reduction of D-lactic acid enantiomer or salt thereof of 90% or more. In specific embodiments, the processes provide reduction of D-lactic acid enantiomer or salt thereof of up to 95%.

In one embodiment, the decomposed organic waste comprises lactic acid derived from a low-pH lactic acid fermentation process.

In certain embodiments, the organic waste comprises a carbohydrate source. In other embodiments, the organic waste is selected from food waste, municipal food waste, residential food waste, agricultural waste, industrial food waste from food and beverages processing facilities, commercial food waste (from hospitals, restaurants, shopping centers, airports etc.), and a mixture or combination thereof. Each possibility represents a separate embodiment.

In further embodiments, the decomposed organic waste is pre-treated prior to step (a). In particular embodiments, pretreatment comprises removal of non-lactic acid-containing impurities.

In additional embodiments, the low-pH lactic acid fermentation process is performed at a pH of about 2 to about 5.5, including each value within the specified range.

In one embodiment, the decomposed organic waste comprises lactic acid derived from decomposition of polylactic acid in an acidic medium.

In certain embodiments, decomposition of polylactic acid in an acidic medium comprises the hydrolysis of polylactic acid in the presence of an acid selected from the group consisting of hydrochloric acid, hydrobromic acid, nitric acid, sulfuric acid, phosphoric acid, and combinations thereof. Each possibility represents a separate embodiment.

In further embodiments, the decomposed organic waste comprises lactic acid by-product that is derived from an industrial production of polylactic acid. In other embodiments, the decomposed organic waste comprises lactic acid by-product that is derived from industrial purification of lactic acid. In yet other embodiments, the decomposed organic waste comprises lactic acid obtained from hydrolyzing lactide by-product from an industrial production of polylactic acid.

In some embodiments, the alkaline substance comprising divalent cations comprises a hydroxide. In another embodiment, the alkaline substance comprising divalent cations is selected from the group consisting of magnesium hydroxide, calcium hydroxide, and a mixture thereof. Each possibility represents a separate embodiment. In one embodiment, the alkaline substance comprising divalent cations is magnesium hydroxide. In further embodiments, the alkaline substance comprising divalent cations comprises a carbonate or a bicarbonate. Each possibility represents a separate embodiment. In further embodiments, the alkaline substance comprising divalent cations is selected from the group consisting of magnesium bicarbonate, magnesium carbonate, calcium bicarbonate, calcium carbonate, and a mixture thereof. Each possibility represents a separate embodiment.

In certain embodiments, the alkaline substance is added in solid form. In alternative embodiments, the alkaline substance is added as an aqueous solution or suspension. In further embodiments, the alkaline substance is gradually added. In other embodiments, the alkaline substance is added as an aqueous solution at a concentration of about 50 to about 500 g/L, including each value within the specified range.

In various embodiments, step (b) and/or (c) are performed at a temperature in the range of about 55° C. to about 100° C., including each value within the specified range. In exemplary embodiments, step (b) and/or (c) are performed at a temperature in the range of about 60° C. to about 100° C., including each value within the specified range. In particular embodiments, step (b) and/or (c) are performed at a temperature in the range of about 70° C. to about 100° C., including each value within the specified range. In yet additional embodiments, step (b) and/or (c) are performed at a temperature in the range of about 80° C. to about 100° C., including each value within the specified range.

In further embodiments, step (b) and/or (c) are performed at a pressure of about 100 to about 1,000 mbar, including each value within the specified range. In other embodiments, step (b) and/or (c) are performed at a mixing speed of about 200 to about 1,000 rounds per minute (RPM), including each value within the specified range.

In some embodiments, precipitation of L-lactate salt in step (c) is performed in a period of time ranging from 10 minutes to 1 day, including each value within the specified range. In additional embodiments, precipitation of L-lactate salt in step (c) is performed in a period of time ranging from 1 hour to 1 day, including each value within the specified range.

In further embodiments, precipitation of L-lactate salt in step (c) is performed at a pH range of about 3 to about 10, including each value within the specified range. In specific embodiments, precipitation of L-lactate salt in step (c) is performed at a pH range of about 4 to about 9, including each value within the specified range. In some embodiments, precipitation of L-lactate salt in step (c) is performed at a pH range of about 5 to about 8, including each value within the specified range. In further embodiments, precipitation of L-lactate salt in step (c) is performed at a pH range of about 6 to about 7, including each value within the specified range.

In other embodiments, the obtained L-lactate salt is separated by filtration or centrifugation. In further embodiments, the obtained L-lactate salt is subjected to subsequent purification. In other embodiments, subsequent purification comprises at least one of crystallization, recrystallization, partitioning, silica gel chromatography, preparative HPLC, and combinations thereof. Each possibility represents a separate embodiment.

In particular embodiments, subsequent purification comprises washing the obtained L-lactate salt, for example using purified water. In other embodiments, subsequent purification comprises dissolving and recrystallizing the obtained L-lactate salt.

In one embodiment, the obtained L-lactate salt comprises less than 6% D-lactate. In another embodiment, the obtained L-lactate salt comprises less than 5% D-lactate. In another embodiment, the obtained L-lactate salt comprises less than 4% D-lactate. In another embodiment, the obtained L-lactate salt comprises less than 3% D-lactate. In another embodiment, the obtained L-lactate salt comprises less than 2% D-lactate. In another embodiment, the obtained L-lactate salt comprises less than 1.5% D-lactate. In specific embodiments, the obtained L-lactate salt comprises less than 1% D-lactate.

In further embodiments, the obtained L-lactate salt is a crystalline L-lactate salt.

In other embodiments, the obtained L-lactate salt is acidified to form L-lactic acid by at least one of hydrochloric acid, hydrobromic acid, nitric acid, sulfuric acid, phosphoric acid, and combinations thereof. Each possibility represents a separate embodiment. In particular embodiments, the L-lactic acid is used for subsequent polylactic acid formation.

Other objects, features and advantages of the present invention will become clear from the following descriptions, examples and drawings.

### DETAILED DESCRIPTION OF THE INVENTION

The present invention provides processes for enantiomeric enrichment of L-lactic acid or salt thereof from an enantiomeric mixture of D-and L-lactic acid derived from recycling procedures. Surprisingly, neutralizing the lactic acid mixture with an alkaline base comprising divalent cations results in the selective precipitation of L-lactate salt thereby providing L-lactic acid/lactate enrichment. The high purity L-lactate salt can further be used for generating new lactic acid-based products.

According to some aspects and embodiments, a process for enriching L-lactic acid enantiomer or salt thereof is provided, the process comprises obtaining a solution comprising an enantiomeric mixture of D-and L-lactic acid; contacting the enantiomeric mixture with an alkaline substance comprising divalent cations; and precipitating L-lactate salt with said divalent cations thereby obtaining L-lactate salt with enriched enantiomeric purity.

According to other aspects and embodiments, a process for producing L-lactate salt with enriched enantiomeric purity is provided, the process comprises obtaining a solution comprising an enantiomeric mixture of D- and L-lactic acid; contacting the enantiomeric mixture with an alkaline substance comprising divalent cations; and precipitating L-lactate salt with said divalent cations thereby obtaining L-lactate salt with enriched enantiomeric purity.

In certain aspects and embodiments, the solution comprising an enantiomeric mixture of D- and L-lactic acid is originated from decomposed organic waste.

As used herein, the term “lactic acid” refers to the hydroxycarboxylic acid with the chemical formula CH₃CH(OH)CO₂H. The terms lactic acid or lactate (unprotonated lactic acid) can refer to the stereoisomers of lactic acid: L-lactic acid/L-lactate, D-lactic acid/D-lactate, or to a combination thereof.

For most industrial applications, L-lactic acid monomers with high purity are required in order to produce polylactic acid (PLA) with suitable properties. Thus, the process of the present invention is directed, in particular, to the production of L-lactate salts with enriched enantiomeric purity, which can be converted to L-lactic acid suitable for reuse without the necessity to eliminate D-lactic acid/lactate monomers.

One advantage stemming from the processes of the present invention is enantiomeric enrichment, which is particularly beneficial for reuse of lactic acid. The enantiomeric enrichment is at least 1%, 2%, 3%, 4%, 5%, 6%, 7%, 8%, 9%, 10% or even more of the initial L-lactic acid content. Each possibility represents a separate embodiment. For example, for an initial enantiomeric mixture containing 90% L-lactic acid and 10% D-lactic acid, a 10% enrichment results in lactate salt containing 99% L-lactate and 1% D-lactate. Within the scope of the present invention is the reduction in the content of D-lactic acid by the process disclosed herein by at least 10%, 15%, 20%, 25%, 30%, 35%, 40%, 45%, 50%, 55%, 60%, 65%, 70%, 75%, 80%, 85%, 90%, 95%, or even 100% of the initial D-lactic acid content. Each possibility represents a separate embodiment. For example, for an initial enantiomeric mixture containing 90% L-lactic acid and 10% D-lactic acid, a 50% reduction in the content of the D-enantiomer results in a lactate salt containing 95% L-lactate and 5% D-lactate. The obtained L-lactate precipitate according to the principles of the present invention comprises less than 6% D-lactate, less than 5% D-lactate, less than 4% D-lactate, less than 3% D-lactate, less than 2% D-lactate, less than 1.5% D-lactate, or less than 1% D-lactate. Each possibility represents a separate embodiment.

According to the principles of the present invention, the enantiomeric mixture of D- and L-lactic acid is derived from decomposed organic waste. For example, the enantiomeric mixture of D- and L-lactic acid may be obtained from the hydrolysis of polylactic acid in the presence of an acid. In another example, the enantiomeric mixture of D- and L-lactic acid may be a by-product of industrial production of polylactic acid or industrial purification of lactic acid. An additional source of lactic acid containing a mixture of the D- and L-enantiomers may be derived as a by-product of hydrolysis of lactide from an industrial production of polylactic acid. Within the scope of the present invention is an enantiomeric mixture of D- and L-lactic acid derived from organic waste that has been subjected to lactic acid fermentation at a low pH.

Organic waste within the scope of the present invention includes, but is not limited to, waste obtained from a variety of sources including food and beverages waste, such as municipal food waste, residential food waste, agricultural waste, industrial food waste from food processing facilities, commercial food waste (from hospitals, restaurants, shopping centers, airports etc.), and a mixture or combination thereof. Each possibility represents a separate embodiment. The organic waste can additionally originate from residues ranging from animal and human excreta, vegetable and fruit residues, plants, cooked food, protein residues, slaughter waste, and combinations thereof. Each possibility represents a separate embodiment. Industrial organic food and beverages waste may include factory waste such as by-products, factory rejects (e.g., expired products, defective products), market returns or trimmings of inedible food portions (such as skin, fat, crusts, and peels). Each possibility represents a separate embodiment. Commercial organic food and beverages waste may include waste from shopping malls, restaurants, supermarkets, etc. Each possibility represents a separate embodiment.

According to certain aspects and embodiments, the organic waste comprises monosaccharides or disaccharides obtained as by-products of sugar production from beet sugar or cane sugar, such as, but not limited to, production of fructose, molasses, or high fructose corn syrup (HFCS). According to other aspects and embodiments, the organic waste comprises starches and starch derivatives such as refined glucose syrups originating from the hydrolysis of starch, which starches may be maize starch, tapioca starch, wheat starch, potato starch, and the like. Each possibility represents a separate embodiment. The organic waste may further be derived from by-products of wine or beer production such as, but not limited to, yeast autolysates and hydrolysates as well as from plant protein hydrolysates, animal protein hydrolysates, and soluble by-products from steeping wheat or maize. Paper sludge hydrolysate obtained by hydrolyzing paper sludge with cellulolytic enzymes may also be used as well as dairy by-products generated during cheese production and dairy beverages production of milk-based beverages including e.g., lactose-free beverages.

According to various aspects and embodiments, the decomposed organic waste comprises a fermentation broth obtained from a fermentation process of a carbohydrate source. When using non-homogenous feedstocks, the decomposed organic waste or fermentation broth typically comprise insoluble organic-based impurities such as, but not limited to, microorganisms (e.g., lactic acid producing microorganisms including e.g. yeasts, bacteria and fungi), fats and oils, lipids, aggregated proteins, bone fragments, hair, precipitated salts, cell debris, fibers (e.g., fruit and/or vegetables peels), and residual unprocessed waste (e.g., food shells, seeds, food insoluble particles and debris, etc.). Each possibility represents a separate embodiment. Non-limiting examples of insoluble inorganic-based impurities include plastics, glass, residues from food packaging, sand, and combinations thereof. Each possibility represents a separate embodiment.

Although not necessary, the decomposed organic waste can further be pre-treated prior to employing the process of the present invention. Suitable pre-treatment includes, but is not limited to, filtration, ultrafiltration, nanofiltration, reverse osmosis (RO) filtration, solvent extraction, repulsive extraction, salt precipitation, crystallization, distillation, evaporation, electrodialysis, and diverse types of chromatography (such as adsorption or ion exchange). Each possibility represents a separate embodiment.

According to the principles of the present invention, the fermentation is performed at a low pH. Due to the formation of L-lactic acid, endogenous lowering of the pH occurs. Thus, in the absence of a neutralizing base, the pH of the fermentation ranges from about 2 to about 5.5, including each value within the specified range.

Typically, fermentation at a low pH can be performed by acid-tolerant (AT) yeast strains including, but not limited to, those belonging to a genus selected from Saccharomyces, Candida, Schizosaccharomyces, Torulaspora, Kluyveromyces, Zygosaccharomyces, and Dekkera. Each possibility represents a separate embodiment.

In other aspects and embodiments, the decomposed organic waste is derived from the acidic hydrolysis of polylactic acid waste. Acidic hydrolysis can be performed in the presence of an acid, for example hydrochloric acid, hydrobromic acid, nitric acid, sulfuric acid, phosphoric acid, and combinations thereof. Each possibility represents a separate embodiment. The hydrolysis may be performed at temperatures in the range of about 30° C. to about 120° C., including each value within the specified range. For example, hydrolysis may be performed at a temperature of about 30° C., 35° C., 40° C., 45° C., 50° C., 55° C., 60° C., 65° C., 70° C., 75° C., 80° C., 85° C., 90° C., 95° C., 100° C., 105° C., 110° C., 115° C. or 120° C. Each possibility represents a separate embodiment. The hydrolysis may be performed at atmospheric pressure or at reduced or elevated pressures, for example at a range of about 100 mbar to about 1,500 mbar, including each value therebetween. Optionally, a catalyst may be added to facilitate hydrolysis.

The decomposed waste may contain various concentrations of D- and L-enantiomers. Typically, the concentration of D- and L-lactic acid is in the range of about 100 to about 1,100 g/L, including each value within the specified range. Exemplary concentrations of D- and L-lactic acid include, but are not limited to, about 100 g/L, 150 g/L, 200 g/L, 250 g/L, 300 g/L, 350 g/L, 400 g/L, 450 g/L, 500 g/L, 550 g/L, 600 g/L, 650 g/L, 700 g/L, 750 g/L, 800 g/L, 850 g/L, 900 g/L, 950 g/L, 1,000 g/L, 1,050 g/L, or 1,100 g/L. Each possibility represents a separate embodiment. The lactic acid enantiomeric ratio in the decomposed waste may vary according to the endogenous content of D-lactic acid as well as racemic lactic acid formation which occurs during fermentation or decomposition. Within the scope of the present invention is decomposed organic waste containing endogenous D-lactic acid in an amount of 10% or less, for example 9%, 8%, 7%, 6%, 5%, 4%, 3% or less. Each possibility represents a separate embodiment. Typically, ratios of D- to L-monomers include, but are not limited, 1:99, 2:98, 3:97, 4:96, 5:95, 6:94, 7:93, 8:92, 9:91, or 10:90. Each possibility represents a separate embodiment. According to some embodiments, the decomposed organic waste contains endogenous D-lactic acid in an amount of at least 3%, for example 4%, 5%, 6%, 7%, 8%, 9%, and up to 10% D-lactic acid. Each possibility represents a separate embodiment.

In case the decomposed organic waste contains particles of PLA waste which have failed to be hydrolyzed or non-lactic acid-containing impurities, they may be separated from the decomposed organic waste, for example by solid-liquid separation techniques such as filtration or decantation. Each possibility represents a separate embodiment.

According to the principles of the present invention, an alkaline substance comprising divalent ions is then added to induce precipitation of L-lactate salt with enriched enantiomeric purity. Suitable alkaline substances include, but are not limited to, hydroxides, carbonates and bicarbonates containing divalent cations such as, but not limited to, magnesium, calcium, or mixtures thereof. Each possibility represents a separate embodiment. Exemplary bases include, but are not limited to, magnesium hydroxide, calcium hydroxide, magnesium bicarbonate, magnesium carbonate, calcium bicarbonate, calcium carbonate, or mixtures and combinations thereof. Each possibility represents a separate embodiment. Currently preferred is the addition of magnesium hydroxide to afford the precipitation of magnesium L-lactate having enriched enantiomeric purity. The alkaline substance can be added in solid form or as an aqueous solution or suspension. Each possibility represents a separate embodiment. When added as an aqueous solution, typically the aqueous solution contains the alkaline substance at a concentration ranging from about 50 to about 500 g/L, including each value within the specified range. Typical concentrations include, but are not limited to, 50 g/L, 60 g/L, 70 g/L, 80 g/L, 90 g/L, 100 g/L, 125 g/L, 150 g/L, 175 g/L, 200 g/L, 225 g/L, 250 g/L, 275 g/L, 300 g/L, 325 g/L, 350 g/L, 375 g/L, 400 g/L, 425 g/L, 450 g/L, 475 g/L, or 500 g/L. Each possibility represents a separate embodiment. In some embodiments, the alkaline substance is gradually added while mixing. It is contemplated that gradual addition of the alkaline substance can be performed in order to avoid excess heating caused by the exothermic reaction.

Mixing speeds throughout the addition of the alkaline substance as well as during precipitation include stirring at about 200 to about 1,000 RPM, including each value within the specified range. Typical mixing speeds include, but are not limited to, 200 RPM, 250 RPM, 300 RPM, 350 RPM, 400 RPM, 450 RPM, 500 RPM, 550 RPM, 600 RPM, 650 RPM, 700 RPM, 750 RPM, 800 RPM, 850 RPM, 900 RPM, 950 RPM, or 1,000 RPM. Each possibility represents a separate embodiment.

In some aspects and embodiments, the alkaline substance is added in stochiometric amounts. In other aspects and embodiments, the alkaline substance is added in excess. In yet other aspects and embodiments, the alkaline substance is added in deficit. According to the principles of the present invention, the alkaline substance is added in an amount of about 50 to about 120 equivalent % based on the number of carboxyl groups present in the enantiomeric mixture. Typical amounts of the alkaline substance include, but are not limited to, 50%, 55%, 60%, 65%, 70%, 75%, 80%, 85%, 90%, 95%, 100%, 105%, 110%, 115%, or 120% equivalents based on the number of carboxyl groups present in the enantiomeric mixture. Each possibility represents a separate embodiment. As will be appreciated by those skilled in the art, any amount which is less than 100 equivalent % based on the number of carboxyl groups present in the enantiomeric mixture refers to deficit amounts of the alkaline substance compared to the lactic acid, and any amount which is more than 100 equivalent % based on the number of carboxyl groups present in the enantiomeric mixture refers to excess amounts of the alkaline substance compared to the lactic acid. Thus, the pH at which precipitation occurs is typically in the range of about 3 to about 10, for example about 4 to about 9, about 5 to about 8, or about 6 to about 7, including each value within the specified ranges. Each possibility represents a separate embodiment.

In some aspects and embodiments, precipitation occurs upon addition of the alkaline substance. Alternatively, precipitation may occur at any time following the addition of the alkaline substance for example at any time duration between about 10 minutes to about 1 day, including each value within the specified range. In various aspects and embodiments, precipitation occurs by forming an initial precipitate and allowing the initial precipitate to mature over a certain period of time. In other aspects and embodiments, precipitation occurs in two steps, first an initial precipitate is formed and separated from the mother liquor, then a second precipitate is formed from the mother liquor and allowed to mature. Typically, precipitation occurs over a period of time ranging from about 1 hour to about 24 hours, for example during the course of 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. Each possibility represents a separate embodiment.

Within the scope of the present invention is the addition of the alkaline substance as well as the precipitation of the L-lactate salt at elevated temperatures. As used herein, the term “elevated temperatures” refers to temperatures which are above room temperatures. Suitable temperatures include temperatures of 40° C. or higher. For example, the temperatures at the steps of adding an alkaline substance and precipitating the L-lactate salt having enriched enantiomeric purity are in ranges of 40° C. to 100° C., 45° C. to 100° C., 50° C. to 100° C., 60° C. to 100° C., 70° C. to 100° C., or 80° C. to 100° C., including each value within the specified ranges. Each possibility represents a separate embodiment. Typically, temperatures in the range of 40° C. to 100° C. are utilized, for example about 40° C., 45° C., 50° C., 55° C., 60° C., 65° C., 70° C., 75° C., 80° C., 85° C., 90° C., 95° C., or 100° C. can be used. Each possibility represents a separate embodiment.

As will be appreciated by those skilled in the art, the duration of precipitation is dependent on the temperature at which precipitation occurs. Thus, in some embodiments, the step of precipitating L-lactate salt is performed at a temperature of about 40° C. for a time duration of about 18 to about 22 hours, including each value within the specified range. In other embodiments, the step of precipitating L-lactate salt is performed at a temperature of about 50° C. for a time duration of about 5 to about 10 hours, including each value within the specified range. In yet other embodiments, the step of precipitating L-lactate salt is performed at a temperature of about 90° C. for a time duration of about 2 to about 5 hours, including each value within the specified range.

The addition of the alkaline substance as well as the precipitation of the L-lactate salt may be performed at various pressures. For example, the pressure at the step of adding an alkaline substance and/or precipitating the L-lactate salt having enriched enantiomeric purity is in the range of about 100 to about 1,000 mbar, including each value within the specified range. Exemplary pressures include, but are not limited to, about 100, 200, 300, 400, 500, 600, 700, 800, 900, or 1,000 mbar. Each possibility represents a separate embodiment.

Typically, the process of the present invention produces a lactate salt with said divalent cations as the end product. However, in order to obtain polylactic acid, the lactate salt may be acidified for subsequent polymerization using e.g., hydrochloric acid, hydrobromic acid, nitric acid, phosphoric acid, sulfuric acid, and a mixture or combination thereof. Each possibility represents a separate embodiment.

The thus obtained L-lactate salt may further be subjected to downstream purification processes. One simple purification that has also surprisingly been found to improve the enantiomeric purity of the L-lactate salt is washing the precipitate, for example using purified water. Within the scope of the present invention are additional purification steps for example crystallization, recrystallization, partitioning, silica gel chromatography, preparative HPLC, and combinations thereof. Each possibility represents a separate embodiment.

The purification processes may include extraction, electrodialysis, adsorption, ion-exchange, crystallization, and combinations of these methods. Several methods are reviewed, for example, in Ghaffar et al. (2014) Journal of Radiation Research and Applied Sciences, 7(2): 222-229; and López-Garzón et al. (2014) Biotechnol Adv., 32(5): 873-904. Alternatively, recovery and conversion of lactic acid to lactide in a single step may be used (Dusselier et al. (2015) Science, 349(6243): 78-80).

Particular downstream purification processes for purifying the enantiomerically enriched lactate salt via crystallization are described in WO 2020/110108 and WO 2022/157768, assigned to the Applicant of the present invention.

The obtained lactate precipitate may be separated from the remaining liquid by microfiltration or nanofiltration. The remaining liquid may undergo concentration, followed by at least one additional precipitation. Following separation of the precipitate from the liquid, the enriched L-lactate salt may be washed with an aqueous solution or with an organic solvent such as ethanol and purified.

While the process disclosed herein is primarily contemplated for enriching L-lactic acid/lactate enantiomer from an enantiomeric mixture comprising D- and L-lactic acid derived from decomposed organic waste, enrichment of the D-enantiomer is also contemplated by the present invention.

Thus, according to certain aspects and embodiments, the present invention provides a process for enriching D-lactic acid enantiomer or salt thereof or producing enantiomerically enriched D-lactate salt, the process comprising the steps of:
(a) obtaining decomposed organic waste comprising an enantiomeric mixture of D- and L-lactic acid;
(b) contacting the enantiomeric mixture with an alkaline substance comprising divalent cations; and
(c) precipitating D-lactate salt with said divalent cations thereby obtaining D-lactate salt with enriched enantiomeric purity.

As used herein and in the appended claims, the term “about” refers to ±10%.

As used herein and in the appended claims, the singular forms “a”, “an”, and “the” include plural references unless the context clearly dictates otherwise. Thus, for example, reference to “an alkaline substance” includes a plurality of such substances unless the context clearly dictates otherwise. It should be noted that the term “and” or the term “or” are generally employed in their sense including “and/or” unless the context clearly dictates otherwise.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Decomposed Organic Waste from a Low-pH Fermentation

Lactic acid fermentation of a carbohydrate source is performed using an acid-tolerant (AT) yeast strain of Saccharomyces cerevisiae in a suitable medium to obtain lactic acid at a concentration of at least 100 g/L. The lactic acid concentration and % of D-lactic acid are measured separately using HPLC. Magnesium hydroxide is then added to the broth to precipitate magnesium L-lactate with enriched enantiomeric purity. The precipitated solid is then filtered off and washed with water to typically afford crude MgLa₂·2H₂O. Optionally, the crude magnesium lactate is dissolved in water followed by its recrystallization.

### Example 2

### Decomposed Organic Waste from Acidic Hydrolysis of PLA

PLA from cafeteria waste is shredded into small pieces and submerged in a 0.01% w/w H₂SO₄ solution, having a pH of ˜2.5. The mixture is heated to 90° C. to facilitate PLA degradation for a time duration of 1-2 days. Optionally, a catalyst is added. pH reduction due to the formation of PLA oligomers and monomers having carboxylic acid end groups is monitored using a pH meter. At the end of the reaction, residual PLA and solid impurities are filtered off using a P3 sintered glass funnel (16-40 μm cut-off). The lactic acid concentration and % of D-lactic acid in the filtered solution are measured separately using HPLC. Magnesium hydroxide is added to the filtered solution to precipitate magnesium L-lactate with enriched enantiomeric purity. The crude magnesium lactate is further purified by subsequent recrystallization.

### Example 3

### LA Enantiomeric Enrichment by Precipitation with Magnesium Hydroxide

A mixture containing L- and D-lactic acid at a D:L ratio of about 4:96 and a concentration of about 45%, was placed in a chemical reactor pre-heated to 50° C. and stirred at 300 RPM. 0.5 molar equivalent of magnesium hydroxide slurry in water (15% w/w) was added dropwise over the course of 0.5 h. The reaction was left to stir overnight at 50° C., during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 43 to 7% due to the precipitation of magnesium lactate, the % D-LA/lactate of total LA/lactate concentration in solution showed an increase from 5.8 to 7.8%. This increase in % D-LA/lactate concentration in solution is indicative of the precipitation of magnesium L-lactate having enriched enantiomeric purity of the L-enantiomer as compared to the initial L-lactic acid content. The results are summarized in the Table 1 below.

TABLE 1

| | % D- |
|---|---|
| | LA/lactate |
| Original LA | 5.8% |
| Mother liquor | 7.8% |
| MgLa₂ precipitate | 4.9% |
| Washed MgLa₂ | 4.1% |
| precipitate | |

### Example 4

### LA Enantiomeric Enrichment by Precipitation with Magnesium Hydroxide at 90° C.

A mixture containing L- and D-lactic acid at a D:L ratio of about 6:94 and a concentration of about 45%, was placed in a chemical reactor pre-heated to 90° C. and stirred at 300 RPM. 0.5 molar equivalent of magnesium hydroxide slurry in water (15% w/w) was added dropwise over the course of 12 min. The reaction was left to stir overnight at 90° C., during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 45 to 13% due to the precipitation of magnesium lactate, the % D-LA/lactate of total LA/lactate concentration in solution showed an increase from 5.7 to 10.5%. This increase in % D-LA/lactate concentration in solution is indicative of the precipitation of magnesium L-lactate having enriched enantiomeric purity of the L-enantiomer as compared to the initial L-lactic acid content. The results are summarized in Table 2 below.

TABLE 2

| | % D- |
|---|---|
| | LA/lactate |
| Original LA | 5.7% |
| Mother liquor | 10.5% |
| MgLa₂ precipitate | 2.0% |
| Washed MgLa₂ precipitate | 1.7% |

### Example 5

### LA Enantiomeric Enrichment by Precipitation with Magnesium Hydroxide at Different Temperatures

A mixture containing L- and D-lactic acid at a D:L ratio of about 6:94 and a concentration of about 45%, was placed in a chemical reactor pre-heated to 40° C. and stirred at 300 RPM. 0.5 molar equivalent of magnesium hydroxide slurry in water (15% w/w) was added dropwise over the course of 26 min. Due to the exothermic nature of the reaction during base addition, the temperature increased to 45° C. The reaction was cooled to 40° C. and left to stir overnight at 40° C., during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 45 to 9% due to the precipitation of magnesium lactate, the % D-LA/lactate of total LA/lactate concentration in solution showed an increase from 5.7 to 7.7%. This increase in % D-LA/lactate concentration in solution is indicative of the precipitation of magnesium L-lactate having enriched enantiomeric purity of the L-enantiomer as compared to the initial L-lactic acid content. The results are summarized in Table 3 below.

TABLE 3

| | % D- |
|---|---|
| | LA/lactate |
| Original LA | 5.7% |
| Mother liquor | 7.7% |
| MgLa₂ precipitate | 4.5% |
| Washed MgLa₂ precipitate | 4.0% |

The procedure was repeated at a temperature of 50° C. and 90° C., and the % reduction of the D-enantiomer was determined. The results indicate that enantiomeric enrichment is enhanced at elevated temperatures which also afford shorter precipitation time durations. The results are summarized in Table 4 below.

TABLE 4

| | Original | % D-LA/lactate in | |
|---|---|---|---|
| | % D- | washed MgLa₂ | precipitation |
| | LA/lactate | precipitate | time |
| 40° C. | 5.7% | 4.0% | 20 h |
| 50° C. | 5.8% | 4.1% | 8 h |
| 90° C. | 5.7% | 1.7% | 3 h |

### Example 6

### LA Enantiomeric Enrichment By Precipitation With Magnesium Carbonate at 50° C.

A mixture containing L- and D-lactic acid at a D:L ratio of about 6:94 and a concentration of about 45%, was placed in a chemical reactor pre-heated to 50° C. and stirred at 300 RPM. 1 molar equivalent of magnesium carbonate slurry in water (33% w/w) was added dropwise over the course of 10 min. During the addition, there was visible CO₂ gas production, indicating the progression of the reaction. The reaction was left to stir overnight at 50° C., during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 45 to 15% due to the precipitation of magnesium lactate, the % D-LA/lactate of total LA/lactate concentration in solution showed an increase from 5.7 to 6.8%. This increase in % D-LA/lactate concentration in solution is indicative of the precipitation of magnesium L-lactate having enriched enantiomeric purity of the L-enantiomer as compared to the initial L-lactic acid content. The results are summarized in Table 5 below.

TABLE 5

| | % D- |
|---|---|
| | LA/lactate |
| Original LA | 5.7% |
| Mother liquor | 6.8% |
| MgLa₂ precipitate | 4.9% |
| Washed MgLa₂ precipitate | 4.8% |

### Example 7

### LA Enantiomeric Enrichment by Precipitation from an Enantiomeric Mixture Containing 9.0% D-lactic Acid

A mixture containing L- and D-lactic acid at a D:L ratio of about 9:91 and a concentration of about 45%, was placed in a chemical reactor pre-heated to 70° C. and stirred at 300 RPM. 0.5 molar equivalent of magnesium hydroxide slurry in water (15% w/w) was added dropwise over the course of 13 min. The reaction was left to stir overnight at 70° C., during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 45 to 9% due to the precipitation of magnesium lactate, the % D-LA/lactate of total LA/lactate concentration in solution showed an increase from 9 to 9.3%. This increase in % D-LA/lactate concentration in solution is indicative of the precipitation of magnesium L-lactate having enriched enantiomeric purity of the L-enantiomer as compared to the initial L-lactic acid content. The results are summarized in Table 6 below.

TABLE 6

| | % D- |
|---|---|
| | LA/lactate |
| Original LA | 9.0% |
| Mother liquor | 9.3% |
| MgLa₂ precipitate | 8.6% |
| Washed MgLa₂ precipitate | 8.1% |

### Example 8

### LA Enantiomeric Enrichment by Precipitation with One-portion Addition of Magnesium Hydroxide to 80% Conversion

A mixture containing L- and D-lactic acid at a D:L ratio of about 3:97 and a concentration of about 35%, was placed in a chemical reactor pre-heated to 40° C. and stirred at 300 RPM. 0.4 molar equivalent of magnesium hydroxide powder was added in one portion. Due to the exothermic nature of the reaction, the temperature increased sharply to 70° C. over the course of 2 min, then gradually cooled down to 50° C. The reaction was left to stir overnight at 50° C., during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 35 to 16% due to the precipitation of magnesium lactate, the % D-LA/lactate of total LA/lactate concentration in solution showed an increase from 2.6 to 3.7%. This increase in % D-LA/lactate concentration in solution is indicative of the precipitation of magnesium L-lactate having enriched enantiomeric purity of the L-enantiomer as compared to the initial L-lactic acid content. The results are summarized in Table 7 below.

TABLE 7

| | % D- |
|---|---|
| | LA/lactate |
| Original LA | 2.6% |
| Mother liquor | 3.7% |
| MgLa₂ precipitate | 1.3% |
| Washed MgLa₂ precipitate | 1.3% |

### Example 9

LA Enantiomeric Enrichment by Precipitation with Magnesium Hydroxide to 90% Conversion

A mixture containing L- and D-lactic acid at a D:L ratio of about 5:95 and a concentration of about 45%, was placed in a chemical reactor pre-heated to 70° C. and stirred at 300 RPM. 0.45 molar equivalent of magnesium hydroxide slurry in water (15% w/w) was added dropwise over the course of 16 min. The reaction was left to stir at 70° C. for 5 h, during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 45 to 11% due to the precipitation of magnesium lactate, the % D-LA/lactate of total LA/lactate concentration in solution showed an increase from 5.6 to 8.7%. This increase in % D-LA/lactate concentration in solution is indicative of the precipitation of magnesium L-lactate having enriched enantiomeric purity of the L-enantiomer as compared to the initial L-lactic acid content. The results are summarized in Table 8 below.

TABLE 8

| | % D- | MgLa₂ |
|---|---|---|
| | LA/lactate | purity |
| Original LA | 5.6% | |
| Mother liquor | 8.7% | |
| MgLa₂ precipitate | 3.7% | 96.3% |
| Washed MgLa₂ precipitate | 2.9% | 96.6% |

### Example 10

### LA Enantiomeric Enrichment by Precipitation with Calcium Hydroxide

A mixture containing L- and D-lactic acid at a D:L ratio of about 5:95 and a concentration of about 45%, was placed in a chemical reactor pre-heated to 70° C. and stirred at 300 RPM. 0.5 molar equivalent of calcium hydroxide slurry in water (15% w/w) was added dropwise over the course of 18 min. The reaction was left to stir overnight at 70° C., though no precipitation occurred during this time. Temperature was decreased to 50° C., after which the calcium lactate precipitated immediately, and the solution solidified completely. The solid was centrifuged at 12,000 g for 5 minutes to extract the mother liquor, then washed with 1 weight equivalent of water by centrifugation. The product was dried at 70° C.

Both total LA/lactate concentration and % D-LA/lactate of the total LA/lactate concentration were measured separately using HPLC. While the overall LA/lactate concentration in the mother liquor decreased from 45 to 10% due to the precipitation of calcium lactate, the % D-LA/lactate of total LA/lactate concentration in the mother liquor showed an increase from 5.0 to 7.7%. This increase in % D-LA/lactate concentration in the mother liquor is indicative of the precipitation of calcium L-lactate having enriched enantiomeric purity of the L-enantiomer as compared to the initial L-lactic acid content. The results are summarized in Table 9 below.

TABLE 9

| | % D- |
|---|---|
| | LA/lactate |
| Original LA | 5.0% |
| Mother liquor | 7.7% |
| CaLa₂ precipitate | 4.7% |
| Washed CaLa₂ precipitate | 4.5% |

### Example 11

### LA Enantiomeric Enrichment by Precipitation with 10% Excess Magnesium Hydroxide

A mixture containing L- and D-lactic acid at a D:L ratio of about 5:95 and a concentration of about 45%, was placed in a chemical reactor pre-heated to 70° C. and stirred at 300 RPM. 0.55 molar equivalent of magnesium hydroxide slurry in water (15% w/w) was added dropwise over the course of 15 min. The reaction was left to stir at 70° C. for 5 h, during which magnesium lactate precipitated. The precipitate was filtered off using a P3 sintered glass funnel (16-40 μm cut-off), washed with 1 weight equivalent of water, and dried at 70° C.

Both total LA/lactate concentration and % D-LA/lactate of the total LA/lactate concentration were measured separately using HPLC. While the overall LA/lactate concentration in solution decreased from 45 to 8% due to the precipitation of magnesium lactate, the % D-LA/lactate of total LA/lactate concentration in solution showed an increase from 5.4 to 9.6%. This increase in % D-LA/lactate concentration in solution is indicative of the precipitation of magnesium L-lactate having enriched enantiomeric purity of the L-enantiomer as compared to the initial L-lactic acid content. The results are summarized in Table 10 below.

TABLE 10

| | % D- | MgLa₂ |
|---|---|---|
| | LA/lactate | purity |
| Original LA | 5.4% | |
| Mother liquor | 9.6% | |
| MgLa₂ precipitate | 2.7% | 96.0% |
| Washed MgLa₂ precipitate | 2.3% | 96.4% |

The results show that while the addition of magnesium hydroxide in 10% excess had very little effect on the purity of the magnesium lactate, a significant enhancement of the enantiomeric purity of the L-enantiomer in the product was obtained.

### Comparative Example 1

### LA Enantiomeric Reduction by Precipitation with Magnesium Hydroxide at 30° C.

A mixture containing L- and D-lactic acid at a D:L ratio of about 6:94 and a concentration of about 45%, was placed in a chemical reactor pre-heated to 30° C. and stirred at 300 RPM. 0.5 molar equivalent of magnesium hydroxide slurry in water (15% w/w) was added dropwise over the course of 1.25 h to avoid overheating. The reaction was left to stir overnight at 30° C., during which magnesium lactate precipitated. The precipitate was filtered off using a P3 sintered glass funnel (16-40 μm cut-off), washed with 1 weight equivalent of water, and dried at 70° C.

The total LA/lactate concentration and % D-LA/lactate of the total LA/lactate concentration were measured separately using HPLC. Both the overall LA/lactate concentration and the % D-LA/lactate of total LA/lactate concentration in solution showed a decrease of from 45 to 14% and from 5.7 to 3.7%, respectively. This decrease in % D-LA/lactate concentration in solution is indicative of the precipitation of magnesium lactate having a decreased enantiomeric purity of the L-enantiomer as compared to the initial L-lactic acid content. The decrease in enantiomeric purity of the L-enantiomer was further enhanced by the wash with 1 weight equivalent of water. The results are summarized in Table 11 below.

TABLE 11

| | % D- |
|---|---|
| | LA/lactate |
| Original LA | 5.7% |
| Mother liquor | 3.7% |
| MgLa₂ precipitate | 7.2% |
| Washed MgLa₂ precipitate | 9.5% |

Thus, adding magnesium hydroxide to a mixture of L- and D-lactic acid at a temperature of 30° C. does not afford enantiomeric enrichment of the L-enantiomer.

### Comparative Example 2

### LA Enantiomeric Reduction by Precipitation from an Enantiomeric Mixture Containing 10.4% D-lactic Acid

A mixture containing L- and D-lactic acid containing 10.4% of the D-enantiomer at a concentration of about 45%, was placed in a chemical reactor pre-heated to 70° C. and stirred at 300 RPM. 0.5 molar equivalent of magnesium hydroxide slurry in water (15% w/w) was added dropwise over the course of 14 min. The reaction was left to stir at 70° C. for 5 h, during which magnesium lactate precipitated. The precipitate was filtered off using a P3 sintered glass funnel (16-40 μm cut-off), washed with 1 weight equivalent of water, and dried at 70° C.

The total LA/lactate concentration and % D-LA/lactate of the total LA/lactate concentration were measured separately using HPLC. Both the overall LA/lactate concentration and the % D-LA/lactate of total LA/lactate concentration in solution showed a decrease of from 45 to 11% and from 10.4 to 9.0%, respectively. The decrease in % D-LA/lactate concentration in solution was comparable to the decrease in total LA/lactate concentration indicating that no change in the enantiomeric purity of the L-enantiomer in the precipitate was achieved. The wash with 1 weight equivalent of water increased the D-enantiomer to L-enantiomer ratio indicating a reduction in the enantiomeric purity of the L-enantiomer in the precipitate. The results are summarized in Table 12 below.

TABLE 12

| | % D- |
|---|---|
| | LA/lactate |
| Original LA | 10.4% |
| Mother liquor | 9.0% |
| MgLa₂ precipitate | 10.4% |
| Washed MgLa₂ precipitate | 11.2% |

Thus, it is contemplated that when an initial mixture containing more than 10% D-lactic acid is used, enantiomeric enrichment of the L-enantiomer cannot be achieved.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-52. (canceled)

53. A process for enriching L-lactic acid enantiomer or salt thereof or for producing enantiomerically enriched L-lactate salt, the process comprising the steps of:
(a) obtaining decomposed organic waste comprising an enantiomeric mixture of D- and L-lactic acid at a concentration of about 100 to about 1,100 g/L, wherein the enantiomeric mixture comprises 10% D-lactic acid or less;
(b) contacting the enantiomeric mixture with an alkaline substance comprising divalent cations in an amount of about 50 to about 120 equivalent % based on the number of carboxyl groups present in the enantiomeric mixture; and
(c) precipitating L-lactate salt with said divalent cations thereby obtaining L-lactate salt with enriched enantiomeric purity as compared to the initial enantiomeric mixture,
wherein step (b) and/or (c) are performed at a temperature in the range of about 40° C. to about 100° C.

54. The process of claim 53 comprising enrichment of L-lactic acid enantiomer or salt thereof by 5% or more, or comprising enrichment of L-lactic acid enantiomer or salt thereof by up to 10%.

55. The process of claim 53, wherein the decomposed organic waste comprises lactic acid derived from a low-pH lactic acid fermentation process; or wherein the decomposed organic waste comprises lactic acid derived from decomposition of polylactic acid in an acidic medium; or wherein the decomposed organic waste comprises lactic acid by-product that is derived from an industrial production of polylactic acid; or wherein the decomposed organic waste comprises lactic acid by-product that is derived from industrial purification of lactic acid; or wherein the decomposed organic waste comprises lactic acid obtained from hydrolyzing lactide by-product from an industrial production of polylactic acid.

56. The process of claim 53, wherein the alkaline substance comprises a hydroxide selected from the group consisting of magnesium hydroxide, calcium hydroxide, and a mixture thereof; or wherein the alkaline substance comprises a carbonate or a bicarbonate selected from the group consisting of magnesium bicarbonate, magnesium carbonate, calcium bicarbonate, calcium carbonate, and a mixture thereof.

57. The process of claim 53, wherein the alkaline substance is added in solid form; or wherein the alkaline substance is added as an aqueous solution or suspension.

58. The process of claim 53, wherein the alkaline substance is added as an aqueous solution at a concentration of about 50 to about 500 g/L.

59. The process of claim 53, wherein step (b) and/or (c) are performed at a pressure of about 100 to about 1,000 mbar; or wherein step (b) and/or (c) are performed at a mixing speed of about 200 to about 1,000 rounds per minute (RPM).

60. The process of claim 53, wherein the obtained L-lactate salt is separated by filtration or centrifugation; and/or wherein the obtained L-lactate salt is subjected to subsequent purification.

61. The process of claim 60, wherein subsequent purification comprises at least one of crystallization, recrystallization, partitioning, silica gel chromatography, and preparative HPLC, or wherein subsequent purification comprises washing the obtained L-lactate salt with purified water.

62. The process of claim 53, wherein the obtained L-lactate salt comprises less than 6% D-lactate; and/or wherein the obtained L-lactate salt is acidified to form L-lactic acid by at least one of hydrochloric acid, hydrobromic acid, nitric acid, phosphoric acid, sulfuric acid, and combinations thereof and used for subsequent polylactic acid formation.

63. A process for enriching L-lactic acid enantiomer or salt thereof or for producing enantiomerically enriched L-lactate salt, the process comprising the steps of:
(a) obtaining decomposed organic waste comprising an enantiomeric mixture of D- and L-lactic acid at a concentration of about 100 to about 1,100 g/L, wherein the enantiomeric mixture comprises 6% D-lactic acid or less;
(b) contacting the enantiomeric mixture with an alkaline substance comprising divalent cations; and
(c) precipitating L-lactate salt with said divalent cations thereby obtaining L-lactate salt with enriched enantiomeric purity as compared to the initial enantiomeric mixture,
wherein step (b) and/or (c) are performed at a temperature in the range of about 50° C. to about 100° C.

64. The process of claim 63 comprising enrichment of L-lactic acid enantiomer or salt thereof by 5% or more, or comprising enrichment of L-lactic acid enantiomer or salt thereof by up to 10%.

65. The process of claim 63, wherein the decomposed organic waste comprises lactic acid derived from a low-pH lactic acid fermentation process; or wherein the decomposed organic waste comprises lactic acid derived from decomposition of polylactic acid in an acidic medium; or wherein the decomposed organic waste comprises lactic acid by-product that is derived from an industrial production of polylactic acid, or wherein the decomposed organic waste comprises lactic acid by-product that is derived from industrial purification of lactic acid, or wherein the decomposed organic waste comprises lactic acid obtained from hydrolyzing lactide by-product from an industrial production of polylactic acid.

66. The process of claim 63, wherein the alkaline substance comprises a hydroxide selected from the group consisting of magnesium hydroxide, calcium hydroxide, and a mixture thereof; or wherein the alkaline substance comprises a carbonate or a bicarbonate selected from the group consisting of magnesium bicarbonate, magnesium carbonate, calcium bicarbonate, calcium carbonate, and a mixture thereof.

67. The process of claim 63, wherein the alkaline substance is added in solid form; or wherein the alkaline substance is added as an aqueous solution or suspension.

68. The process of claim 63, wherein the alkaline substance is added as an aqueous solution at a concentration of about 50 to about 500 g/L.

69. The process of claim 63, wherein step (b) and/or (c) are performed at a pressure of about 100 to about 1,000 mbar, or wherein step (b) and/or (c) are performed at a mixing speed of about 200 to about 1,000 rounds per minute (RPM).

70. The process of claim 63, wherein the obtained L-lactate salt is separated by filtration or centrifugation; and/or wherein the obtained L-lactate salt is subjected to subsequent purification.

71. The process of claim 70, wherein subsequent purification comprises at least one of crystallization, recrystallization, partitioning, silica gel chromatography, and preparative HPLC, or wherein subsequent purification comprises washing the obtained L-lactate salt with purified water.

72. The process of claim 63, wherein the obtained L-lactate salt comprises less than 3% D-lactate; and/or wherein the obtained L-lactate salt is acidified to form L-lactic acid by at least one of hydrochloric acid, hydrobromic acid, nitric acid, phosphoric acid, sulfuric acid, and combinations thereof and used for subsequent polylactic acid formation.
